(19) United States
(12) Patent Application Publication
(10) Pub. No.: US 2006/0003435 A1
(43) Pub. Date: Jan. 5, 2006

(54) Production by yeasts of aspartic proteinases from plant origin with sheep's, cow's, goat's milk, etc. clotting and proteolytic activity

(76) Inventors: Maria Salome Soares Pais, Lisboe (PT); Filomena da Conceicao Sousa Soares Calixto, Moita (PT); Rudy J. Planta, Amsterdam (NL); Keatie Henriette Ouborg Planta, Amsterdam (NL)

Correspondence Address: Finnegan, Henderson, Farabow,;Garrett & Dunner, L.L.P., 901 New York Avenue, N.W., Washington, DC, 20005-3315, US

(21) Appl. No.: 11/097,381

(22) Filed: Apr. 4, 2005

Related U.S. Application Data
(63) Continuation of application No. 09/980,819, filed on May 23, 2002, now abandoned.

(30) Foreign Application Priority Data
Jun. 9, 1999 (PT) 102318 B

(51) Int. Cl. C12N 1/16; C12N 1/18

(52) U.S. Cl. 435/254.2

(57) ABSTRACT

This invention is valid for recombinant enzymes produced from transformed yeast with coding genes for plant-origin aspartic acid proteinases. These proteinases have considerable sheep's, cow's and goat's milk clotting activity. They can be produced in large quantities by cultivating transformed yeast in a liquid medium. They are secreted into the culture medium and can be supplied in liquid or lyophilised form. The activity of these enzymes is similar to that of chymosin (an animal-origin enzyme) used in the production of cheese on an industrial scale. Recombinant aspartic acid proteinases differ from chymosin in their casein cleavage capacity. Recombinant plant enzymes cleave α, β and κ caseins. Chymosin only cleaves κ casein. The ability of plant-origin recombinant aspartic acid proteinases to cleave α, β and κ caseins is responsible for the special flavour, smell and consistency of the cheese produced.

### BACKGROUND OF THE INVENTION

1. Field of the Invention

The use of a yeast expression system has become a way of producing large quantities of different types of compounds on an industrial scale. Regarding the production of plant-origin aspartic acid proteinases with industrial applications, there has not been any news of yeast expression with regard to production for use on an industrial scale.

The object of this invention patent, described below, refers to the construction of plasmids, the transformation of yeast strains and the production of plant-origin aspartic acid proteinases. These proteins are proteolytic and milk clotting enzymes which can be used in the cheese production and other biotechnological applications

2. Description of the Prior Art

Plant aspartic proteinases have been isolated, characterised and cDNA have been prepared since 1997 (D'Hondt et al, 1997). The studies with the aspartic proteinases derived from Cynara cardunculus named Cyprosins started in the nineties, with the purification of the enzymes (at that time known as Cynarases, Heimgartner et al, 1990), followed in 1992 with their partial characterisation. The construction of a cDNA library and the isolation of a cDNA clone was first reported in 1993 (Cordeiro 1993) and published in several journals since 1994, together with the characterisation of their tissue specificity (Brodelius et al, 1995; Cordeiro et al, 1994; 1994; Cordeiro et al, 1995). The sequence of the CYPRO11 cDNA was included in the gene bank and reported later on (Brodelius et al, 1998). Purification of Cardosins, the other group of Cynara cardunculus aspartic proteinases, was achieved in 1995 (Faro et al, 1995). After this, an extensive work was performed with respect to some biochemical properties including specificity towards substrates (Faro et al, 1995; Verissimo et al, 1995, 1996). Characterisation and partial protein sequence analysis started in 1995 (Faro et al, 1995; Verissimo et al, 1996). Since then, the studies performed in further characterisation of the enzymes, their glycosylation pattern (Costa et al, 1997), their histological and cytological location (Ramalho-Santos et al, 1997) and function (Faro et al, 1998) have been published. The characterisation of the enzyme precursor (Ramalho-Santos et al, 1998a) and identification of its proteolytic processing mechanism (Ramalho-Santos et al, 1998b) helped to understand the molecular and physiological relevance of the intra-molecular domains such as the pro-sequence and the plant-specific-insert. Crystallisation studies on the structure of Cardosin A started in 1998 (Bento et al, 1998) and has contributed to the knowledge of intramolecular aspects related to the biological function (Frazão et al, 1999). Only very recently the cDNA encoding the Cardosin A was cloned. Functional aspects of protein domains and motifs and further implications in the function of this enzyme were better clarified (Faro et al, 1999).

The description of a DNA construct for expression of polypeptides by yeast cells was prior reported (EP 0123289). The constructs employed the entire yeast α-factor secretion leader. Since then the production of several polypeptides of interest have been reported in yeast cells, including aspartic proteinases from animal origin, as for example bovine chymosin (Mellor et al, Gene 1983, 24: 1-14), and human cathepsin E (Yamada et al, Biochimica et Biophysica Acta 1994, 1206: 279-285).

### EXPERIMENTAL

Construction of Plasmids. Transformation of Yeast Strains and Production of Plant Aspartic Proteinases

The insertion of coding gene CYPRO11 into a plant-origin proteinase constitutes the experimental model for controlling the yeast expression of plant-origin aspartic acid enzymes.

Two Escherichia coli-yeast expression system vectors were constructed, using a type 2μ multi-copy plasmid and a centromeric plasmid having a low number of copies. The choice of gene used was the leucine deficient one (LEU2). The expression cassette contained developer Gal7 promotor and four different leader sequences upstream from the heterologous gene. Transcription of the heterologous gene was stopped by a PGK1 terminator.

From the different leader sequences tested (native prosequence, preSUC2-proCYPRO11, preMFα-proCYPRO11 and preproMFα), we concluded that preMFα-proCYPRO11 was the best leader sequence for the production of plant-origin aspartic acid proteinases, whether cyprosins corresponding to the plant-origin model proteins coded by gene CYPRO11, or other commercially interesting plant-origin acidic aspartic proteinases.

The MFα yeast presequence is sufficient to develop secretion of the aspartic acid proteinase into the culture medium, and the use of a prosequence of the gene is not necessary. The native prosequence was essential to the active protein's production.

The use of centromeric plasmids having a low number of copies gave better results than type 2μ multi-copy plasmids.

Different yeast strains were tested, including Saccharomyces cerevisae BJ1991 (MATα leu2 trpl ura3-52 prbl-1122 pep4-3), BJ2168 (MATα leu2 trpl ura3-52 prcl-1122 pep4-3), MT302/1c-a (arg5-6 leu2-12 his3-11 his3-15 peb4-3 ade1), W303-1^{a} (MATα leu2-3,112 ura3-1 trpl-1 his3-11,15 ade2-1 can1-100 GAL SUC2).

These strains were kept on YPD agar plates containing 1% yeast extract, 2% bacto-peptone, 2% glucose and 1.5% agar.

The transformed yeast was grown in an SD medium (0.67% yeast nitrogen base without amino acids, DIFCO, 2% (w/v) glucose), supplemented with amino acids suited to the auxotrophic needs of each strain, except for the leucine one.

The cultures were collected and washed once with sterile distilled water. The cells were resuspended in a YPGal medium (1% yeast extract, 2% bacto-peptone, 4% galactose) and used to inoculate the same medium at a density of A₆₀₀=0.2. The cultures were incubated in the same culture conditions until they reached densities of A₆₀₀=2, 6 or 10.

Of the yeast strains tested, protease deficient strain BJ1991 produced and secreted into the culture medium the largest quantities of aspartic acid proteinase with considerable milk clotting and proteolytic activity. The secretion of proteolytic enzymes was therefore dependent on culture growth. The recombinant proteinase with the highest degree of clotting and proteolytic activity was obtained in the stationary phase of the YPGal medium's growth (A₆₀₀=10). In the exponential phase (A₆₀₀=2), the yeast cells secreted an inactive recombinant proteinase having a high molecular weight. It was considered to be an unprocessed form of the proteinase in which a specific region of the genes of plant-origin acidic aspartic proteinases called a specific plant insert had not been removed.

The largest sub-unit of the recombinant proteinases secreted by the yeast was glycosilated, in the only site possible for glycosilation, and contained a considerable number of manose type glycan chains.

Preparation of Polyclonal Antibodies

The total proteic extract used to produce polyclonal antibodies against plant-origin acidic aspartic proteinase with considerable coagulation and proteolytic activity was obtained from the dry flowers of Cynara cardunculus by maceration in a mortar in liquid nitrogen and extraction with 50 mM of Tris HCI buffer at a pH of 8.3 (Heimgartner et al., 1990). The proteins were fractionated in 12% SDS-PAGE using 100 μg of total protein extract per well. The gel was tinted with a 0.02% Commassie Blue solution in distilled water. The bands corresponding to the largest sub-unit of the plant enzyme (31-32.5 kDa in the SDS-PAGE gel) were isolated and the content of each well was sent to EUROGENTEC (Belgium) for the production of antibodies.

Isolation of the Plant-Origin Proteinase and Western Blotting Analysis

Isolation of the recombinant plant-origin proteinase from the cell extracts was done using 30 ml of yeast cells grown to densities of A₆₀₀=2, 6 or 10. After collection, the cells were washed with distilled water, resuspended in 500 μl of buffer and exploded by shaking them with glass balls.

Isolation of the recombinant proteinase from the culture medium was done after collecting the medium and concentrating it almost 10 times by ultracentrifugation.

The proteinase concentration was ascertained using the Bio-Rad protein analysis kit in accordance with the manufacturer's instructions. 50 μg of total proteic extract from the yeast cells or 1.125 g of the concentrated culture medium was analysed in 12% SDS-PAGE. The proteins were transferred to a nitro-cellulose membrane (Bio-Rad) using Trans-Blot SD Semi-Dry Electrophoretic Transfer Cell (Bio-Rad) equipment in accordance with the manufacturer's instructions. Proteins were detected using polyclonal antibody CCMPI prepared in accordance with the description in the previous section and Boeringer Mannheim's Chemiluminescence Western Blotting Kit in accordance with the manufacturer's instructions.

The results obtained showed that the transformed yeast produces plant-origin aspartic acid proteinase and that the inactive form is found in cells in the exponential growth phase while the active form is secreted into the culture medium. This peculiarity is decisive when it comes to getting good performance for the extraction and purification of plant-origin acidic aspartic proteinases produced from yeast.

Analysis of the Plant-Origin Recombinant Enzyme's Clotting and Proteolytic Activity

Proteolytic activity was analysed in accordance with the Twinning method (1984). The casein preparation marked with isothiocyanate (casein-FTC) was made in accordance with the author's instructions. The reactive mixture contained 30 μl of 0.2M sodium citrate buffer, pH 5.1, 20 l of casein-FTC and 20 μl of enzyme solution (3 μg/μl in the case of total proteic extract from the yeast cells or 150 ng/μl in the case of concentrated culture medium).

Two control tests were done by replacing the enzymatic solution with the reactive buffer. Another control was performed by using the same yeast strain transformed with the same plasmids in which the heterologous gene was absent. The samples were incubated at 37° C. for 30 minutes. Reaction was stopped by adding 120 μl of 5% trichloracetate acid (TCA) in all but one of the controls. In the latter case, the same amount of 0.5M Tris HCI buffer at a pH of 8.0 (positive control) was added. The samples were centrifuged and a 150 μl aliquot of the supernatant fraction was diluted to 3 ml with 0.5M Tris HCI buffer at a pH of 8.5. The control (without enzymes), whose reaction was stopped with the TCA solution, was used to ascertain the formation of soluble fluorescent compounds in TCA with enzyme intervention. Relative fluorescence of the samples was ascertained using wavelengths of 490 nm for excitation and 525 nm for emission in a Shimadzu RF-1501 (Shimadzu Corporation, Kyoto, Japan) spectrofluorimeter. The percentage of relative fluorescence (% RF) was calculated by subtracting the negative control values from the values, and considering the positive control values as 100% RF. For statistical analysis of the results, each sample had three replicas and three independent readings were taken. The data obtained were analysed with the Student's t test (α=0.05). Greatest proteolytic activity, obtained for the best combination/yeast strain, was 15% RF/μ of protein. This figure refers to standard culture conditions, and can be increased under conditions optimised for industrial purposes namely using mutant yeast strains chosen for their maximum recombinant proteinase secretion into the culture medium.

Ascertaining Clotting Activity

Clotting activity was ascertained in test tubes, using unconcentrated culture medium in accordance with the following method: 10 ml of the culture medium of the transformed YPGal yeast cells was added to 3 ml 12% of skimmed milk (bacto-Difco) and 100 ml mM CaCl₂. The pH of the culture medium for the culture grown to either A₆₀₀=6 or 10 was approximately 5.0. For the culture medium of the culture grown to A₆₀₀=2, the pH was adjusted to 5.0 using HCI. The samples were kept at 37° C. until the onset of coagulation. The coagulation was evident.

1. A method for producing an aspartic proteinase from plant origin using yeast as a host cell said method comprising the introducing into that host cell a DNA construct containing the sequence encoding the said aspartic proteinase from plant origin and growing said host cell comprising said DNA construct containing the sequence in a culture medium whereby said aspartic proteinase from plant origin or part thereof is secreted or not into the culture medium

2. A method according to claim 1 whereby said DNA sequence forms part of a DNA construct which is introduced into said host cell and which comprises in the direction of transcription a pro sequence heterologous to said host cell or to said aspartic proteinase from plant origin and said pro-sequence is joined in reading frame to the said DNA sequence coding for the mature aspartic proteinase from plant origin whereby said aspartic proteinase from plant origin is secreted by said host cell

3. A method according any one of claims 1 and 2 wherein said aspartic proteinase from plant origin is a plant enzyme

4. A method according to 1 to claim 3, wherein said enzyme is a plant aspartic proteinase or an unprocessed form thereof

5. A method according to claim 1 to 4 wherein said enzyme is cyprosin or mutant forms thereof

6. A method according to any one of the claims 1 to 4 wherein said aspartic proteinase from plant origin is cardosin or mutant forms thereof

7. A method according to any one of claims 1 to 6 wherein said host cell is an yeast strain with laboratory or industrial interest

8. A method according to any one of claims 1 to 7 wherein said host cell is from the genus Saccharomyces used for the transformation and expression of plant aspartic proteinases encoding genes and the secretion of the aspartic proteinase from plant origin encoded by said genes or secretion of part of said aspartic proteinase from plant origin

9. A transformed yeast host cell comprising an expression cassette which comprises, in the direction of transcription a leader sequence functional in said host cell composed of a pro-sequence heterologous to said host cell or to an aspartic proteinase from plant origin and said pro-sequence is joined in reading frame to the DNA sequence encoding for the said mature aspartic proteinase from plant origin

10. A cell according to claim 9 wherein said pro-sequence is a plant aspartic proteinase pro-sequence

11. A cell according to claims 9 and 10 wherein said aspartic proteinase is a plant aspartic proteinase or a thereof

12. A cell according to claim 9, 10 and 11 wherein said aspartic proteinase from plant origin is cyprosin or an unprocessed form thereof

13. A cell according to claim 9, 10, 11 and 12 wherein said aspartic proteinase from plant origin is cardosin or an unprocessed form thereof

14. The expression cassettes constructs for use in a yeast host cells comprising: in the direction of transcription a leader sequence composed of a pro-sequence heterologous to said host cell or to aspartic proteinase from plant origin and said pro-sequence is joined in reading frame to the DNA sequence encoding for the said mature aspartic proteinase from plant origin

15. The expression cassettes constructs according to claim 14, wherein said pro-sequence is heterologous to said host cell or to said aspartic proteinase from plant origin or to said host cell and said aspartic proteinase from plant origin

16. The expression cassettes constructs according to claim 14 or 15 further comprising the pro-sequence of the plant aspartic proteinase and the plant gene encoding plant aspartic proteinases

17. A method according to any one of claims 1 to 8 wherein said aspartic proteinase from plant origin or part thereof is isolated either from the cell extracts or from the culture medium

18. A method for detection of the aspartic proteinase from plant origin either in the cell extracts or in the culture medium using the antibody raised against the said aspartic proteinase from plant origin

19. A method for detection of the aspartic proteinase from plant origin either in the cell extracts or in the culture medium using the antibody CCMP1

20. The transformed yeast cells in culture described in claims 9 to 13 characterised by their production of recombinant plant aspartic proteinases with milk clotting activity which cleave caseins from milk of different origins, namely sheep's, cow's and goat's milk confirmed by milk clotting tests

21. The transformed yeast cells in culture described in claims 9 to 13 characterised by their production of recombinant plant aspartic proteinases including cyprosins and cardosins capable of giving to cheese a special taste and flavour
